(19) United States
(12) Patent Application Publication
(10) Pub. No.: US 2016/0174887 A1
(43) Pub. Date: Jun. 23, 2016

(54) SYSTEM AND METHOD FOR EXTRACTING PHYSIOLOGICAL INFORMATION AND MEDICAL INSTRUMENT FOR USE IN THE SYSTEM

(71) Applicant: KONINKLIJKE PHILIPS N.V., Eindhoven (NL)

(72) Inventors: Ihor KIRENKO, Veldhoven (NL); Mukul ROCQUE, Eindhoven (NL)

(21) Appl. No.: 14/959,465

(22) Filed: Dec. 4, 2015

(30) Foreign Application Priority Data
Dec. 18, 2014 (EP) 14198738.8

(51) Int. Cl. A61B 5/1455; A61B 5/1459; A61B 5/1495; A61B 5/00; A61B 5/0295; A61B 5/026

(52) U.S. Cl. CPC A61B 5/14552; A61B 5/0295; A61B 5/0261; A61B 5/1495; A61B 5/7278; A61B 5/1459; A61B 5/6848; A61B 5/6852; A61B 2562/0238

(57) ABSTRACT

The present invention relates to system for extracting physiological information indicative of at least one vital sign of a subject from detected light transmitted through a part of the subject (1). To overcome the limitations of the contactless camera-based reflective PPG technique and classical contact sensors the proposed system comprises a medical instrument (12, 24) for at least partly introduction into a subject's body, one or more light sources (14, 15) for emitting light, said one or more light sources being mounted to the part of the medical instrument that is introduced into the subject's body during use, a light detection unit (16) for detecting light emitted by said one or more light sources and transmitted through a part of the subject, and a processing unit (18) for deriving physiological information indicative of at least one vital sign from the detected light using remote photo-plethysmography. Said light detection unit (16) comprises an imaging unit, in particular a camera, for acquiring a sequence of images of skin areas of the subject over time. Said processing unit (18) is configured for deriving physiological information indicative of at least one vital sign from said sequence of images using remote photo-plethysmography.

### CROSS-REFERENCE TO PRIOR APPLICATION

This application claims the benefit of European Patent Application No. 14198738.8 filed Dec. 18, 2014. The application is hereby incorporated by reference herein.

### FIELD OF THE INVENTION

The present invention relates to a system and a corresponding method for extracting physiological information indicative of at least one vital sign of a subject from detected light transmitted through a part of the subject, such as a person (e.g. a patient) or animal. Further, the present invention relates to a medical instrument for use in such a system.

### BACKGROUND OF THE INVENTION

Vital signs of a person, for example the heart rate (HR), the respiration rate (RR) or the arterial blood oxygen saturation (SpO2), serve as indicators of the current health state of a person and as powerful predictors of serious medical events. For this reason, vital signs are extensively monitored in inpatient and outpatient care settings, at home or in further health, leisure and fitness settings.

One way of measuring vital signs is plethysmography. Plethysmography generally refers to the measurement of volume changes of an organ or a body part and in particular to the detection of volume changes due to a cardio-vascular pulse wave traveling through the body of a subject with every heartbeat.

Photoplethysmography (PPG) is an optical measurement technique that evaluates a time-variant change of light reflectance or transmission of an area or volume of interest. PPG is based on the principle that blood absorbs light more than surrounding tissue, so variations in blood volume with every heart beat affect transmission or reflectance correspondingly. Besides information about the heart rate, a PPG waveform can comprise information attributable to further physiological phenomena such as the respiration. By evaluating the transmittance and/or reflectivity at different wavelengths (typically red and infrared), the blood oxygen saturation (SpO2) can be determined.

Modern photoplethysmography sensors do not always provide fast and reliable measurement in critical situations. For instance, contact finger pulse oximeters (based on transmissive PPG with source/detector geometry on either side of tissue to be measured) are vulnerable to motion of a hand, and fails in case of centralization of a patient due to lower blood volumes on body peripherals. Contact forehead pulse oximeter sensors (using a reflective PPG with source and detectors on the same side as tissue to be measured) are supposed to be more robust to a centralization effect. However, the accuracy, robustness and responsiveness of a forehead sensor depends heavily on correct positioning of a sensor on a forehead and proper pressure applied to a skin (too tight application of a sensor might reduce a local blood pulsatility, too loose application might lead to non-reliable measurements due to motion artifacts and/or venous pulsatility). Moreover, conventional contact pulse sensors are obtrusive, and their wires might hinder a workflow.

Recently, non-contact, remote PPG (rPPG) devices (also called camera rPPG devices) for unobtrusive measurements have been introduced. Remote PPG utilizes light sources or, in general radiation sources, disposed remotely from the subject of interest. Similarly, also a detector, e.g., a camera or a photo detector, can be disposed remotely from the subject of interest. Therefore, remote photoplethysmographic systems and devices are considered unobtrusive and well suited for medical as well as non-medical everyday applications. This technology particularly has distinct advantages for patients with extreme skin sensitivity requiring vital signs monitoring such as Neonatal Intensive Care Unit (NICU) patients with extremely fragile skin or premature babies.

Verkruysse et al., “Remote plethysmographic imaging using ambient light”, Optics Express, 16(26), 22 Dec. 2008, pp. 21434-21445 demonstrates that photoplethysmographic signals can be measured remotely using ambient light and a conventional consumer level video camera, using red, green and blue color channels.

Wieringa, et al., “Contactless Multiple Wavelength Photoplethysmographic Imaging: A First Step Toward “SpO2 Camera” Technology”, Ann. Biomed. Eng. 33, 1034-1041 (2005), discloses a remote PPG system for contactless imaging of arterial oxygen saturation in tissue based upon the measurement of plethysmographic signals at different wavelengths. The system comprises a monochrome CMOS-camera and a light source with LEDs of three different wavelengths. The camera sequentially acquires three movies of the subject at the three different wavelengths. The pulse rate can be determined from a movie at a single wavelength, whereas at least two movies at different wavelengths are required for determining the oxygen saturation. The measurements are performed in a darkroom, using only one wavelength at a time.

Although such reflective remote PPG technique is the most convenient, in cases of severe disturbance from ambient illumination, or if the pulsatility of a PPG signal is very low, SNR of the extracted signal will be weak and the measurements would be vulnerable to artifacts. The reason for this is the fact that pulsatility of a PPG signal measured in reflective mode is lower than the one measured in a transmissive mode, due to a smaller penetration depth of a light in a reflective mode. Especially, pulsatility of a PPG signals measured in red and infrared color range using a remote reflective PPG approach might be very low, thus reducing the accuracy of SpO2 measurements. Another advantage of transmission mode measurements is that the measurement path is the same for all channels which improves artifact removal techniques. Moreover, a reflective non-contact way of PPG measurements suffers from specular reflectance depending on the orientation of the illumination and camera. This can cause further errors in the measurement of PPG based vital signs.

Justin P. Phillips et al., “Evaluation of a Fiber-optic Esophageal Pulse Oximeter”, 31st Annual International Conference of the IEEE EMBS, Minneapolis, Minn., USA, Sep. 2-6, 2009, discloses a dual-wavelength fiber-optic pulse oximetry system for the purposes of estimating oxygen saturation from the esophagus. A probe containing miniature right-angled glass prisms is used to record PPG signals from the esophageal wall. Signals were recorded successfully in 19 of 20 patients, demonstrating that PPG signals could be reliably obtained from an internal vascularized tissue site such as the esophageal epithelium. The results demonstrate that SpO2 may be estimated in the esophagus using a fiber-optic probe.

U.S. Pat. No. 7,376,451 B2 discloses a non-intrusive physiological data measurement system and method, as well as an optically induced treatment system. The measurement system includes a monitoring mechanism that includes light emitter modules capable of emitting light at at least two wavelengths. The light emitted from the light emitter modules is transmitted through a subject and to a light receiving mechanism, such as an optical sensor.

Physiological data is taken from the received light. The system also can ascertain movement of the subject by obtaining an initial outline of the subject and comparing that outline with a subsequently obtained outline. A therapeutic optic system includes a non-adhering light emitting mechanism for providing light at therapeutic wavelengths.

EP 2 666 412 A1 discloses a method for obtaining diagnostic information relating to the lungs of a subject that includes directing into tissue of the lungs of the subject light of a first wavelength and detecting part of the light that has passed primarily through microcirculatory tissue of the lungs and generating a signal which is a function of intensity of the detected light. The signal is then processed to derive a PPG curve for pulmonary microcirculatory arteries. The method is implemented using various locations for a light source and a detector, including various combinations of positioning on the thoracic wall, and insertion into the esophagus. Use of two PPG curves for different wavelengths in the infrared range allows derivation of mixed venous blood oxygen saturation.

U.S. Pat. No. 5,005,573 A discloses an endotracheal breathing tube for use in surgical operations that is equipped with a light emitting device adjacent its distal end to reside within the patient's trachea during use and with a compatible photosensitive detector positionable outside the patient's body in contact with the neck to intercept light transmitted from the light emitting device for performing accurate oximetry measurements and calculations of the patient's arterial blood oxygen saturation. In one embodiment, a light emitting diode is affixed to the distal portion of the tube body. In another embodiment, an optical fiber extends from a light emitting diode outside the patient's body through the tube to a fiber terminus at the distal location on the tube body.

U.S. Pat. No. 4,898,175 A discloses an out-body observing apparatus, in which an illuminating light fed by an illuminating device is emitted from the tip part of an insertable part of an endoscope inserted into a body cavity and is radiated onto a part to be observed. This illuminating light having passed through a living body tissue is imaged by an imaging device provided outside the body. This imaging means delivers a picture image signal to a signal processing device. This signal processing device processes the signal and outputs a video signal to a displaying device. This displaying device displays on a picture surface the image of the observed part of the tissue within the living body.

### SUMMARY OF THE INVENTION

It is an object of the present invention to provide a system and a corresponding method for extracting physiological information that overcomes the limitations of the contactless camera-based reflective PPG technique and classical contact sensors. It is a further object of the present invention to provide a medical instrument for use in such a system.

In a first aspect of the present invention a system is presented comprising:
a medical instrument for at least partly introduction into a subject's body,
one or more light sources for emitting light, said one or more light sources being mounted to the part of the medical instrument that is introduced into the subject's body during use,
a light detection unit for detecting light emitted by said one or more light sources and transmitted through a part of the subject,
a processing unit for deriving physiological information indicative of at least one vital sign from the detected light using remote photo-plethysmography,
wherein said light detection unit comprises an imaging unit, in particular a camera, for acquiring a sequence of images of skin areas of the subject over time, and
wherein said processing unit is configured for deriving physiological information indicative of at least one vital sign from said sequence of images using remote photo-plethysmography.

In a further aspect of the present invention a method is presented comprising:
detecting light emitted by one or more light sources and transmitted through a part of the subject, said one or more light sources being configured to emit light and being mounted to a part of a medical instrument that is introduced into the subject's body during use,
deriving physiological information indicative of at least one vital sign from the detected light using remote photo-plethysmography
wherein said detecting of light comprises acquiring a sequence of images of skin areas of the subject over time, and
wherein said deriving of physiological information comprises deriving physiological information indicative of at least one vital sign from said sequence of images using remote photo-plethysmography.

In a yet further aspect of the present invention a medical instrument is presented comprising:
an instrument body for at least partly introduction into a subject's body, and
one or more light sources for emitting light, said one or more light sources being mounted to the part of the medical instrument that is introduced into the subject's body during use.

Preferred embodiments of the invention are defined in the dependent claims. It shall be understood that the claimed method can have similar and/or identical preferred embodiments as the claimed system and as defined in the dependent claims.

The present invention is based on the idea to combine the advantages of transmissive mode for PPG signal acquisition and contactless sensing using a light detection unit. Transmissive PPG assumes location of a light source and light sensors at opposite sides of a tissue or a body part (such as used in a finger clip pulse oximeter sensor). According to the present invention one or more light sources are embedded into a medical instrument, such as a feeding tube for a neonate, and a light detection unit, e.g. comprising one or more cameras, as a light sensor.

For instance, a feeding tube is present within the body of a neonate to deliver nutrients to the neonate. Attaching light sources to such a feeding tube would not cause any extra burden to a neonate. In this embodiment, the light at one or more (various) wavelengths (e.g. red and/or near infrared (NIR) light) will travel through the part of the body of the neonate and will be sensed by a light detection unit, thus providing a transmissive PPG signal. Since the feeding tube is more central to the body than external contact probes used conventionally on the fingers or the toes for contact PPG, the measurement will be more immune to cases of centralization and will be more responsive.

Instead of a feeding tube other medical instruments may be used, such as a catheter, an endoscope, a needle, a breathing hose or, generally, any medical instrument that is at least partly introduced in the subject's body. Further, even medical implants provided with one or more light sources may be used.

The present invention further exploits the advantages of a large sensing area, achieved particularly with camera-based measurements, and a relatively high SNR of a PPG signal extracted in a transmissive mode.

Contrary to the fiber-optic esophageal pulse oximeter described in the above mentioned paper of Justin P. Phillips et al., which represents a contact reflective PPG single spot sensor, the system and method according to embodiments of the present invention disclose a contactless measurement approach for transmissive PPG at multiple wavelengths, which is preferably configured to obtain spatial two dimensional (2D) information from the subject's surface (skin area). The prior art fiber-optic esophageal pulse oximeter has several major disadvantages, mainly based on the fact that a single spot reflective PPG sensor assumes a certain path of a dedicated light, which depends on the structure and thickness of neighboring tissue. For instance, the distance between a photosensor and a light source of a contact sensor are fixed and the SpO2 calibration values are selected specifically for such optical path and skin location. Therefore, the accuracy and robustness of such sensors are very sensitive to its exact placement. Hence, the fiber-optic esophageal pulse oximeter is very sensitive to exact positioning of a fiber optic, which is very difficult to control in practice.

Another problem refers to the shielding of an optical path. Contact reflective sensors are designed in such way that a photosensor should not see the scattered light from its own light source or from ambient light, but only the light, which penetrated to the tissue and modulated by blood volume pulse. This cannot be guaranteed by such a fiber-optic esophageal pulse oximeter since the exact location of a sensor-light pair inside the esophagus is not controllable and the size of esophagus might vary.

All these disadvantages are overcome by the system and method according to the present invention. The contactless light detection unit generally acquires a signal from a relatively large 2D area (thus avoiding the dependency on the exact optical path of a contact reflective probe) and sees only the dedicated light, which went thought the tissue, but no scattered or specularly reflected light. Moreover, by analyzing the 2D distribution of intensity of the dedicated light, the location of a light source(s) inside the subject's body, e.g. inside the esophagus, can be estimated.

Compared to the system described in U.S. Pat. No. 7,376,451 B2 the system and method according to embodiments of the present invention guarantees that the whole light, which is produced by the one or more dedicated light sources passes through the body area of more or less equal thickness, whereas in the known system the illumination patch through the body is much worse determined For instance, a part of the light can pass directly from a mattress to a camera, or go through various parts of the body with different body structure and thickness (e.g. back, legs, etc.). Thus, the proposed system and method provide a much better reproducibility of the signal strength due to the better determined location of a light source within the subject's body.

Further, the length of the light path from the internal light source(s) to the light detection unit is smaller than through the whole body so that less light power is needed. This also improves a safety aspect, since in the known system a lot of near infrared illumination needs to be produced, part of which might reach the eyes of the subject (typically a baby).

Still further, the robustness to motion can be achieved easier with the proposed system and method, since the motion of a body would not change the light path between the internal light sources and the detection unit significantly. In contrast, within the known system during motion a part of light emitted from the light sources within the mattress can get lost.

According to a preferred embodiment said light detection unit comprises one or more photo detectors or an imaging unit, in particular a camera. Generally, the light detection unit may be configured for being mounted to the subject's body during use, but preferably the light detection unit is arranged remotely from the subject. In another embodiment said one or more light sources, e.g. LEDs, are configured for emitting light in at least two wavelengths or wavelength ranges, preferably in the red and/or infrared wavelength range. This is particularly useful for obtaining physiological information about the oxygen saturation of arterial blood (SpO2).

Generally, said one or more light sources may emit light continuously. In a preferred embodiment they are configured for emitting light periodically. The light detection unit is then preferably configured to detect light during first periods in which said one or more light sources do not emit light and during second periods in which said one or more light sources emit light, and the processing unit is the preferably configured to derive physiological information from light detected during both periods. This enables an improvement of the robustness of the acquisition of vital signs, e.g. obtained from a combined analysis of the reflective and transmissive measurement or by using an estimation of the ambient illumination from measurements during the first periods.

Still further, in an embodiment the system further comprises a plurality of light sources distributed over a part of the medical instrument. This enables an improvement of the reliability of the acquisition of vital signs by a combined analysis of vital signs independently obtained from different measurement locations. Further, the processing unit may then be configured to generate the pulse-transit-time of the blood through part of the subject's body.

In still another embodiment said light detection unit comprises an imaging unit, in particular a camera, for acquiring a sequence of images of skin areas of the subject over time and said processing unit is configured for deriving physiological information indicative of at least one vital sign from said sequence of images using remote photo-plethysmography and for deriving respiratory information from said sequence of images. Respiratory information may e.g. be obtained by monitoring and evaluating movements of the chest wall or belly are of a person caused by the respiration.

In another embodiment the processing unit is configured to adapt calibration constants of SpO2 calculations depending on the DC value of the light sensed by the light detection unit. The DC level of light intensity depends on the optical patch, in particular the optical structure and thickness of the subject's tissue between the light sources and the light detection unit. This further improves the accuracy of the SpO2 measurements.

### BRIEF DESCRIPTION OF THE DRAWINGS

These and other aspects of the invention will be apparent from and elucidated with reference to the embodiment(s) described hereinafter. In the following drawings

FIG. 1 shows a schematic diagram of an embodiment of a system according to the present invention and

FIG. 2 shows an embodiment of a medical instrument according to the present invention.

### DETAILED DESCRIPTION OF THE INVENTION

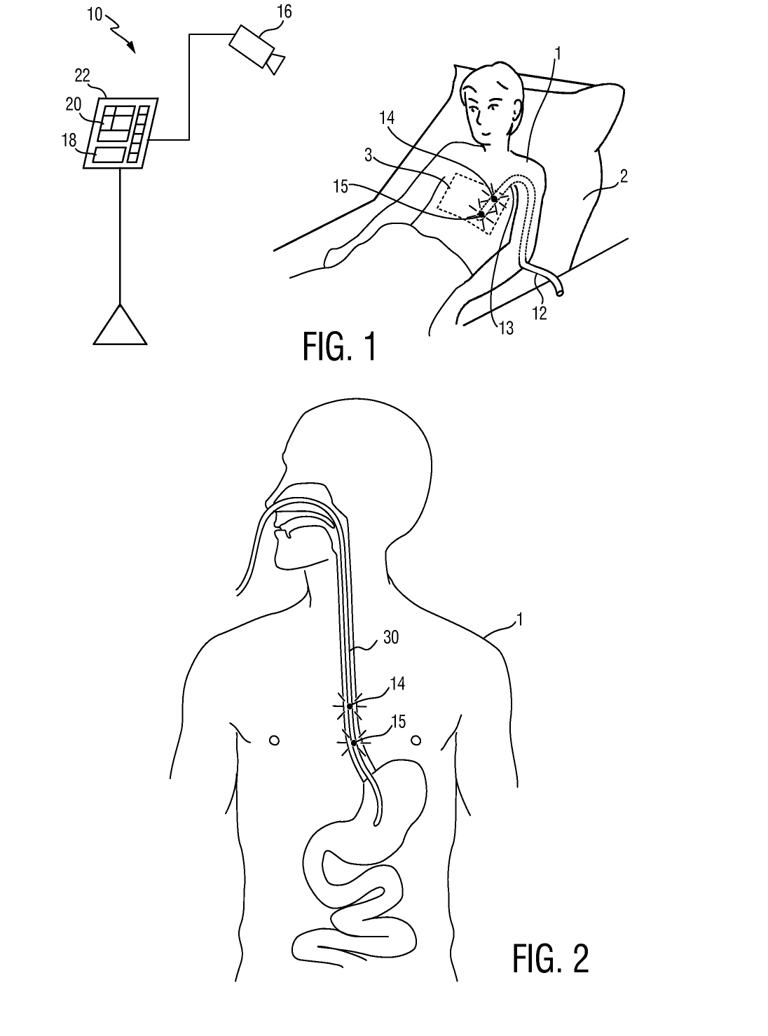

FIG. 1 shows a schematic diagram of an embodiment of a system 10 according to the present invention for extracting physiological information indicative of at least one vital sign of a subject from detected light transmitted through a part of the subject 1. The subject 1, in this example a patient, lies in a bed 2, e.g. in a hospital or other healthcare facility, but may also be a neonate or premature infant, e.g. lying in an incubator, or person at home or in a different environment, such as a nursing home, or even an animal.

The system 10 comprises a medical instrument 12 for at least partly introduction into a subject's body. In this exemplary embodiment the medical instrument 12 is a catheter or endoscope, which is partly introduced into the patient's body, e.g. through a blood vessel.

The system 10 further comprises one or more light sources 14, 15 for emitting light. Said one or more light sources, which may e.g. be LED or laser diodes, are mounted to the part of the medical instrument that is introduced into the subject's body during use. In this exemplary embodiment two LEDs 14, 15 are mounted to the instrument body 13, which is introduced into the patient's body, in this example to the tip portion of the medical instrument 12. Other than shown in FIG. 1, the one or more light sources 14, 15 may also be mounted to or integrated into the foremost part of the medical instrument 12.

The system 10 further comprises a light detection unit 16 for detecting light emitted by said one or more light sources 14, 15 and transmitted through a part of the subject 1. In this exemplary embodiment the light emitted by the light sources 14, 15 transmits through the tissue of the chest and through the chest wall so that it can be detected by the light detection unit that detects light from at least the skin area corresponding to the chest area.

The system 10 further comprises a processing unit 18 for deriving physiological information indicative of at least one vital sign from the detected light using remote photo-plethysmography. For instance, from the detected light over time photo-plethysmography (PPG) signals can be derived which are e.g. directly indicative of the patient's heart beat.

The light detection unit 16 may comprise one or more photo detectors or image sensors, but preferably comprises a camera (also referred to as imaging unit, or as camera-based or remote PPG sensor) including a suitable photosensor for (remotely and unobtrusively) capturing image frames of the subject 1. The camera particularly acquires a sequence of image frames of the subject 1 over time, from which PPG signals can be derived. The image frames captured by the camera may particularly correspond to a video sequence captured by means of an analog or digital photosensor, e.g. in a (digital) camera. Such a camera usually includes a photosensor, such as a CMOS or CCD sensor, which may also operate in a specific spectral range (visible, IR) or provide information for different spectral ranges. The camera may provide an analog or digital signal. The image frames include a plurality of image pixels having associated pixel values. Particularly, the image frames include pixels representing light intensity values captured with different photosensitive elements of a photosensor. These photosensitive elements may be sensitive in a specific spectral range (i.e. representing a specific color). The image frames include at least some image pixels being representative of a skin portion of the subject. Thereby, an image pixel may correspond to one photosensitive element of a photo-detector and its (analog or digital) output or may be determined based on a combination (e.g. through binning) of a plurality of the photosensitive elements.

The system may further comprise an interface 20 for displaying the determined information and/or for providing medical personnel with an interface to change settings of the system 1 or any elements thereof. Such an interface 20 may comprise different displays, buttons, touchscreens, keyboards or other human machine interface means. The processing unit 18 and the interface 20 may be implemented as a common device 22, such as a computer.

A system 11 as illustrated in FIG. 1 may, e.g., be located in a hospital, healthcare facility, elderly care facility or the like. Apart from the monitoring of patients, the present invention may also be applied in other fields such as neonate monitoring. The uni- or bidirectional communication between the various elements of the system 1 may work via a wireless or wired communication interface. Other embodiments of the present invention may include a device 22 or processing unit 18, which is not provided stand-alone, but integrated into the detector 16, e.g. into a camera.

FIG. 2 shows a first embodiment of a medical instrument 24 according to the present invention, which is a feeding tube in this embodiment.

A feeding tube, also known as a gavage tube, is used to give nutrition to patients, such as infants, who cannot eat on their own. The gavage tube is normally used in a hospital, but it can also be used in a home to feed infants. The tube can also be used to give medication to a patient. In most of the situations at NICU, a feeding tube is inserted all the time, while the baby is in an incubator, thus reducing the stress caused by removal and insertion of a tube.

In an embodiment of the present invention, miniature dedicated light source(s) 14, 15 are placed inside the feeding tube 30. The light detection unit 16 is preferably aligned in time and in spectrum range with the miniaturized light sources 14, 15 embedded in the feeding tube 24. The dedicated light source 14, 15 can emit light with at least one narrow spectrum band in the range >700 nm, e.g. for extraction of a heart rate signal from the detected light. In case SpO2 shall be measured as vital sign, the light sources 14, 15 allow emitting of light in at least two wavelength ranges, preferably a first range of 650 nm-750 nm and a second range of >800 nm. The light at the two wavelength ranges can be emitted either sequentially in time or simultaneously.

In another embodiment of the present invention, the light sources operating at at least two wavelengths are distributed over the length of the medical instrument, e.g. of the feeding tube. Thus, the light detection unit 16, particularly a camera, acquires a 2D PPG image of a large part of the subject's body. Another application of such an embodiment may be in the estimation of pulse transit time (PTT) based blood pressure measurement of the subject. The estimation of PTT can be done by measuring the time distance between beats detected by the same camera from regions of interest located along the tube at some distance from each other.

In yet another embodiment of the present invention, a video signal from a camera (used as light detection unit) is used not only for acquisition of PPG signals, but also for estimation of motion of a chest or belly area of the subject's body in order to improve the accuracy and motion robustness of extracted PPG signals and to measure respiratory signals from said motions of the chest or belly area.

In yet another embodiment of the present invention, the light sources embedded in the medical instrument are providing the light not continually, but periodically. In this embodiment, the frame rate of the light detection unit is twice large than the rate of the dedicated illumination. In those periods, when the dedicated light of the light sources is off, the light detection unit acquires PPG signal generated by reflected (ambient) illumination, i.e. from light reflected from the skin of the person in response to ambient illumination. Therefore, in this embodiment, the same light detection unit acquires PPG signals in contactless reflective mode (from ambient illumination) and in transmissive mode (from dedicated light source(s) embedded in the medical instrument).

In yet another embodiment of the present invention, the system improves the robustness of transmissive PPG measurements by combined analysis of the signals acquired with the dedicated light (from the one or more light sources 14, 15) on and off. During time periods when the light sources 14, 15 are off, the amount of ambient illumination can be estimated and, assuming a relatively slower change in ambient illumination, can be compensated for. Furthermore, since the subject's vital signs such as heart rate and SpO2 would not change based on the method of measurement, the combined analysis of signals acquired in transmission mode and reflective mode are preferably used to improve robustness of the measurement.

In still another embodiment the light detection unit 16 is not arranged remotely and in a contactless manner with respect to the subject, e.g. in the form of a camera, but is mounted to the subject's body. For instance, a photo sensor or image sensor may be mounted to the subject's chest by use of a sticky tape or a belt. Optimally, the light detection unit 16 is mounted in the area of the body in which the light sources are placed within the body so that as much light emitted by the light sources transmitting the tissue can be detected. For instance, a light detector mounted on a chest of a person can be a part of a wearable sensing device, e.g. accelerometer (used for measurement of respiration and body motion), or skin temperature measurement patch.

The proposed method and system can be used for continuous monitoring of vital signs of neonates in NICU. Moreover, it can be also used for continuous monitoring of vital signs or PTT of an adult patient in ICU, which e.g. uses a feeding tube.

SpO2 measurement requires calibration constants which vary for different body parts. Moreover, during centralization, the pulsatility of PPG signal as well as the responsiveness of SpO2 measurement (speed of SPO2 changes) would vary significantly across a body. According to the present invention, the source of the signal, i.e. the location of the light sources within the subject's body, remains the same (with respect to the subject, as long as the medical instrument is not moved). Thus, a fixed calibration constant can be used and the exact detection/recognition of body parts is not required.

While the invention has been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. System for extracting physiological information indicative of at least one vital sign of a subject from detected light transmitted through a part of the subject, said system comprising:
a medical instrument for at least partly introduction into a subject's body,
one or more light sources for emitting light, said one or more light sources being mounted to the part of the medical instrument that is introduced into the subject's body during use,
a light detection unit for detecting light emitted by said one or more light sources and transmitted through a part of the subject,
a processing unit for deriving physiological information indicative of at least one vital sign from the detected light using remote photo-plethysmography,
wherein said light detection unit comprises an imaging unit, in particular a camera, for acquiring a sequence of images of skin areas of the subject over time, and
wherein said processing unit is configured for deriving physiological information indicative of at least one vital sign from said sequence of images using remote photo-plethysmography.

2. System as claimed in claim 1,
wherein said light detection unit is configured for being mounted to the subject's body during use.

3. System as claimed in claim 1,
wherein said light detection unit is arranged remotely from the subject.

4. System as claimed in claim 1,
wherein said one or more light sources are configured for emitting light in at least two wavelengths or wavelength ranges.

5. System as claimed in claim 1,
wherein said one or more light sources are configured for emitting light periodically.

6. System as claimed in claim 5,
wherein said light detection unit is configured to detect light during first periods in which said one or more light sources do not emit light and during second periods in which said one or more light sources emit light and
wherein said processing unit is configured to derive physiological information from light detected during both periods.

7. System as claimed in claim 1,
comprising a plurality of light sources distributed over a part of the medical instrument.

8. System as claimed in claim 7,
wherein said processing unit is configured to generate the pulse-transit-time of the blood through part of the subject's body.

9. System as claimed in claim 1,
wherein said medical instrument is a feeding tube, catheter, endoscope, needle or breathing hose.

10. System as claimed in claim 1,
wherein said processing unit is configured to adapt calibration constants of SpO2 calculations depending on the DC value of the light sensed by said light detection unit.

11. Method for extracting physiological information indicative of at least one vital sign of a subject from detected light transmitted through a part of the subject, said method comprising:
detecting light emitted by one or more light sources and transmitted through a part of the subject, said one or more light sources being configured to emit light and being mounted to a part of a medical instrument that is introduced into the subject's body during use,
deriving physiological information indicative of at least one vital sign from the detected light using remote photo-plethysmography,
wherein said detecting of light comprises acquiring a sequence of images of skin areas of the subject over time, and
wherein said deriving of physiological information comprises deriving physiological information indicative of at least one vital sign from said sequence of images using remote photo-plethysmography.

12. Medical instrument for use in a system as claimed in claim 1 for extracting physiological information indicative of at least one vital sign of a subject from detected light transmitted through a part of the subject, said medical instrument comprising:
an instrument body for at least partly introduction into a subject's body, and
one or more light sources for emitting light, said one or more light sources being mounted to the part of the medical instrument that is introduced into the subject's body during use.
